(19) United States
(12) Patent Application Publication
(10) Pub. No.: US 2012/0310307 A1
(43) Pub. Date: Dec. 6, 2012

(54) TREATMENT OF FUNGAL INFECTION BY LIGHT IRRADIATION

(76) Inventor: Bo Zhou, Larchmont, NY (US)

(21) Appl. No.: 13/476,006

(22) Filed: May 20, 2012

Related U.S. Application Data
(60) Provisional application No. 61/492,357, filed on Jun. 1, 2011.

(51) Int. Cl. A61N 5/06

(52) U.S. Cl. 607/88

(57) ABSTRACT

Described herein are the systems and methods of treating diseases related to fungal infection with light therapy. In one embodiment, an apparatus that utilizes one or multiple light emitting diodes (LED) to treat the fungus is applied externally to the infection area. Light therapy may applied periodically at scheduled times with continuous or pulsed radiation.

Continuation of Application No. 61/492,357, Jun. 1, 2011

### FIELD OF INVENTION

The invention pertains to the systems and methods of treating diseases related to fungal infection.

### BACKGROUND

Fungal infections represent the invasion of tissues by one or more species of fungi. Most fungal infections occur due to the human exposure to a source of fungi in the nearby environment, such as the air, soil, or bird droppings. The common diseases caused by fungal infection includes finger nail and toe nail fungus, Athlete's foot, jock itch, scalp and hair infection, ringworm, fungal sinus infection, barber's itch and others. Most of time, those disease causes pain, discomfort and social embarrassment to the patients. Sometimes it may even cause permanent damage and in some cases eventually be fatal to certain patients, such as organ transplant recipients and HIV/AIDS Carrier.

Onychomycosis is one example of diseases caused by fungal infection. It is a chronic fungal nail infection that affects approximately 10% of the population^{[1,2]}. The prevalence of onychomycosis has increased dramatically during the last few decades, and is usually higher among certain groups, such as the elderly, patients with diabetes, and immunocompromised individuals^{[3,4]}. Incidence of fungal infection in adults over age 60 can be as high as 14-28%^{[3]}. In patients with diabetes or immunocompromised disease, onychomycosis increases the risk of recurrent cellulitis and ulceration. Dystrophic nails may predispose patients to secondary bacterial infections. Without treatment toenails can become thick, causing pressure, irritation, and pain. One clinical study has found that: among 150 subjects with onychomycosis, 54% subjects reported toenail discomfort and 36% reported pain associated with walking which limit physical mobility and activity^{[5]}. In addition to pain and potential increase of health risk, onychomycosis also impact patients' quality of life and cause psychosocial problems. Based on a survey done by Drake L A etc^{[6]}, as many as 74% of onychomycosis patients felt social embarrassment related to the disease. Anxiety, depression, loss of self-esteem and confidence, avoidance of intimacy, and impaired relationships are among the negative impacts reported. An effective treatment is needed to benefit the patients from both physical and psychosocial perspectives.

Treatment of onychomycosis is still a challenge for physicians. The efficacy of current treatment options, including topical, oral, mechanical and chemical therapies or a combination of these modalities, remains disappointing. Topical drug treatment for onychomycosis has low efficacy because the topical drugs are typically unable to penetrate the hyperkeratotic nail plate. As a result, a therapeutically sufficient quantity of drug cannot be delivered to the sites of fungal infection. In addition, rapid recurrence of symptoms is often observed after discontinuing use of the drug^{[7]}. Although oral antifungal agents have some improved efficacy, they post risk of side effects^{[8]}. There is a significant risk of liver toxicity, prolonged loss of taste, and life-threatening drug interactions. The development of fungal resistance to oral antifungal agents in long-term use also poses concern. Another treatment modality is invasive nail surgery, which is a very traumatic procedure^{[9]}. Topically applied antifungal drugs may work somewhat better after removing the nail plate by surgery or chemical dissolution. However, this procedure leaves the patient without a nail for months, increase risks of postoperative infections, and is often ineffective^{[10]}.

### BRIEF SUMMARY OF INVENTION

Described herein are the systems and methods of treating diseases related to fungal infection with light therapy. In one embodiment, an apparatus that utilizes one or multiple light emitting diodes (LED) to treat the fungus is applied externally to the infection area. Light therapy may applied periodically at scheduled times with continuous or pulsed radiation.

### BRIEF DESCRIPTION OF THE FIGURES

FIG. 1 illustrates the killing rate of blue light on T. Rubrum at different exposure time. Power density of the blue light was 2.4 W/cm².

FIG. 2 shows the killing rate of red light on C. Albicans at different exposure time. Power density of the red light was 2.4 W/cm².

FIG. 3 is a schematic of one embodiment of treatment module, where the light source is physically secured onto the target area.

### DETAILED DESCRIPTION OF THE INVENTION

The invention disclosed herein relates to the system and method of a non-invasive treatment approach by using light radiation to treat fungal infected tissues. These treatments are suitable for treating fungal diseases occurred on the surface of the body, in skin folds and nails. It uses light radiation at certain wavelength to cause the death or retarded growth of fungal pathogens residing in human tissue. Reactive Oxygen Species (ROS) can be generated under light-tissue/fungi interaction. Light at certain wavelength has high efficiency in stimulating generation of ROS in fungal infected areas. Higher power (as compared to traditional low light therapy which has power ranged in mw) of light radiation and prolonged exposure time on tissue creates a fatal concentration of ROS which is toxic to the pathogen, resulting the retardation or death of the fungi^{[12]}. Under the same circumstance of radiation, health human tissue around the infected area is not significantly affected by the light. Depends on the scattering and absorption in the tissue, light can penetrate the human skin or nails and reach the infection tissue in the range of mm to cm. This makes it possible to effectively treat fungi infection underneath the skin or nail plate as compared to the use of topical drugs. Light radiation only affects local tissue within the radiation zone and has no toxicity to the whole body, which is superior to the current oral treatment agents, such as Lamisil.

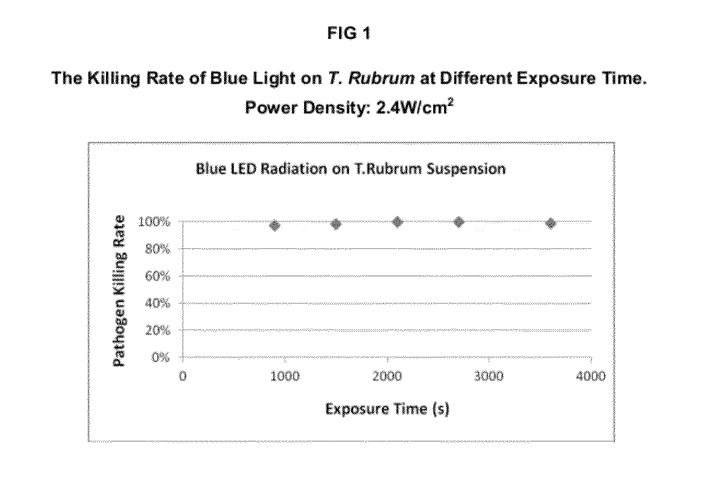

Dermatophytes (including the genera Trichophyton, Epidermophyton and Microsporum) are by far the most common pathogens of onychomycosis, with Trichophyton Rubrum (T. Rubrum) causing 80% of the infections^{[11]}. Candida Albicans (C. Albicans) are another common pathogen in fungal disease. Ex vivo studies on liquid suspension of these fungi have demonstrated the effectiveness of blue and red light radiation in causing retarded growth or death of the fungi. FIG. 1 demonstrates the effective killing rate of the blue light radiation on T. Rubrum. The killing rates on T. Rubrum are plotted against the exposure time. The blue LED light has center wavelength at 470 nm and power density at 2.4 W/cm². Samples of the liquid T. Rubrum culture were aliquoted into selected wells of 96-well tissue culture plates for radiation with predefined dosage. The exposure time varies from 15 to 60 minutes. After radiation, liquid culture samples were diluted and spread onto separated plates and incubated at 37° C. for 72 hours. Samples from the same liquid culture without light exposed were diluted and incubated at the same condition as positive control. After incubation, colonies were counted manually. The killing rate was calculated based on the decrease of colony-forming unit (CFU) counts after irradiation divided by the CFU counts of control (no irradiation under same condition). The killing rate on T. Rubrum is above 95% in all tested samples with radiation time ranging from 15 to 60 minutes.

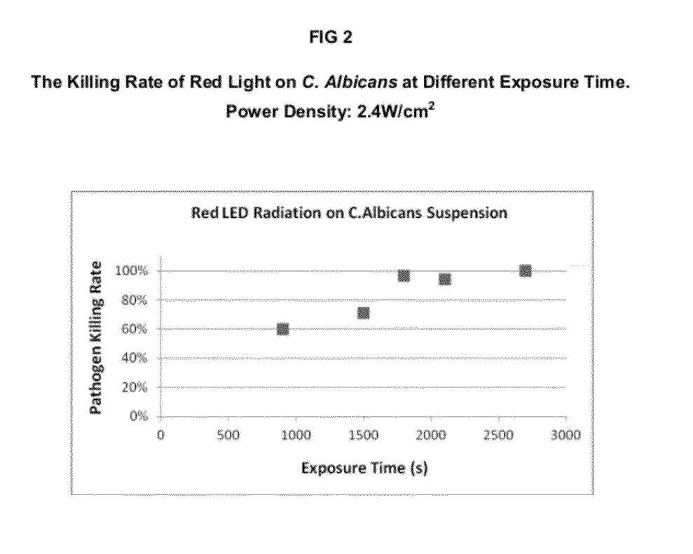

Although not as efficient as blue light, red light (center wavelength at approximately 630 nm) also demonstrate a certain level of effectiveness in killing T. Rubrum. On the other side, red light is more effective in killing C. Albicans. FIG. 2 shows the killing rate of red light on C. Albicans. The preparation and experiment procedure are similar to the T. Rubrum test described above.

Herein, we disclose a non-invasive approach that delivers light energy at the specific wavelength to cause the retardation or death of fungi which infect the human body.

Fungal infected area will be radiated with light at certain wavelength(s) depends on the type of pathogens, such as visible light at a range of 400 to 500 nm or at a range of 600 to 700 nm, with sufficient light exposure time and power density, such as exposure time of 1 to 200 minutes and power density of 0.1 to 10 W/cm². If necessary, adaptations to limit photon or thermal related damage to non-target tissues can be used. Equipment such as temperature sensors, thermal imaging systems and light control systems that monitor the treatment, e.g., position of the light, level of cooling, contact of cooling device with treatment surface, duration and dosage of light energy at the treatment site, temperature of the target site on the surface or within deep tissues can be incorporated. Contact or non-contact cooling systems for surgical application are similarly known in the art, and are useful in combination with the approaches described herein. These all provide methods for controlling the radiation of light in both the fungal-infected tissues and the non-target tissues. Another means of modulating light radiation in treatment area is to use periodic pulsing of the light.

One embodiment of the apparatus which deliver the light therapy could consist of three modules described below:
1. the Treatment module which could include
   a. One or multiple light sources such as blue or red LED(s) or laser(s) to generate light at specific wavelength(s)
   b. A delivery system that can position and secure treatment sites such as toes or fingers or skin under light radiation during the treatment
   c. If necessary, a cooling system to maintain skin, nail or other human body temperature to avoid tissue burn or other heat related side effects (such as pain, etc)
   d. Any other necessary components to ensure the effectiveness and safety of light therapy, such as temperature sensing and feedback system, body motion sensing and feedback system, etc.
2. the Control module which could include
   a. A control panel to manage the treatment mode, time and power, surface cooling and other necessary component to control the electronic parts
   b. A display panel for displaying necessary information during the treatment such as time, power density, temperature and others.
   c. If necessary, a control program responding to feedback system of such as temperature, body motion or other sensing technique implanted in the treatment module.
3. The Power Supply module which provides powers for the light source, cooling fans, and other electronic parts.

One or multiple blue or red LEDs may be adapted in this design. Various methodologies could be applied to maintain the surface temperature, such as an “air cooling” device which blows room temperature or cold air onto the treatment area, or a “contact cooling” system which has a cooled heat exchanger in contact with the surface.

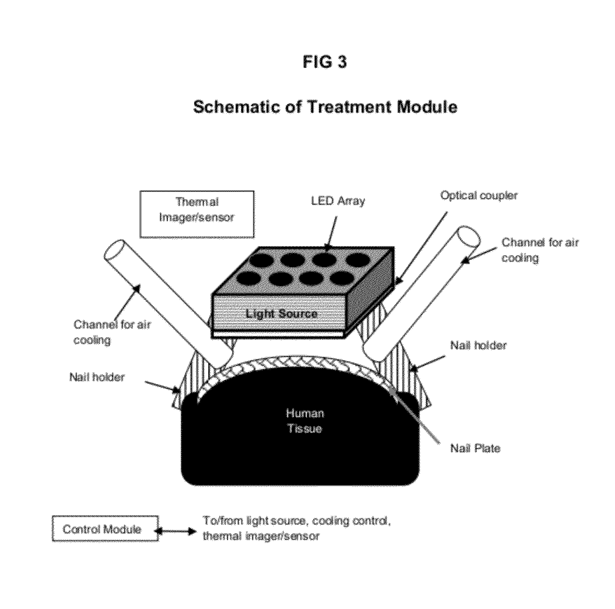

FIG. 3 illustrates one embodiment of treatment module, where the light source is physically secured onto the target area. The treatment system includes a light source and an associated delivery assembly, a tissue mounting assembly, a controller, a cooling assembly and optionally, a temperature device. In the illustrated embodiment of FIG. 3, the light source includes an array of LED emitters with an associated delivery assembly, in the form of beam-forming optical couplers. In other embodiments, a different form and number of light sources can be used.

The illustrated optional temperature device is in the form of a temperature sensor, which generates a signal representative of the patient's tissue temperature based on the thermal footprint of the treatment area. Other forms of generating a temperature signal can be used in other embodiments, including a processor which generates estimates of the temperature of the treatment tissue and adjacent tissue, based on a thermal model of the patient and the energy applied to and extracted from the treatment tissue, directly or indirectly.

The optional cooling assembly is in the form of a cooler blowing room-temperature or cold air through channels for thermal convection to sufficiently cool a portion of the patient's treatment region. In various embodiments, the contact heat exchanger may be adapted to extract heat across the patient's tissue by a liquid heat transfer agent passing through a contact plate, by a thermoelectric heat transfer device or another known form of controlled surface contact cooling device.

The light source and associated delivery assembly, the temperature device (and its generated temperature signal) and the cooling assembly, are all coupled to the control module. Those elements operate under the control of control module to control the application of the light via beams to (and optionally extraction of excessive heat across surfaces from) the treatment area of the patient whereby the temperature of the tissue is below approximately 40° C. throughout the whole treatment period.

The device above can be used in conjunction with current treatments modalities, such as topical, mechanical and oral treatments.

### REFERENCE

1. Roberts D T. Prevalence of dematophyte onychomycosis in the United Kingdom: results of an omnibus survey. Br J Dermatol 1992: 126: 23.
2. Gupta A K, Jain H C, Lynde C W, Macdonald P, Cooper E A, Summerbell R C. Prevalence and epidemiology of onychomycosis in patients visiting physicians' offices: a multicenter Canadian survey of 15,000 patients. J Am Acad Dermatol 2000: 43: 244-248.
3. Gupta A K, Jain H C, Lynde C W. Prevalence and epidemiology of unsuspected onychomycosis in patients visiting dermatologists' offices in Ontario, Canada—a multicenter survey of 2001 patients. Int J Dermatol 1997: 36: 783-787.
4. Alteras I, Saryt E. Prevalence of pathogenic fungi in the toe-webs and toe-nails of diabetic patients. Mycopathologia 1979:67(3): 157-159.
5. Schein J R, Gause D, Stier D M, et al. Onychomycosis: baseline results of an observational study. J Am Podiatr Med Assoc 1997: 87: 512-519.
6. Drake L A, Scher R K, Smith E B, et al. Effect of onychomycosis on quality of life. J Am Acad Dermatol 1998: 38,5(1):702-704.
7. Finch J J, Warshaw E M. Toenail onychomycosis: current and future treatment options. Dermatol Ther 2007;20:31-46.
8. Katz H I. Drug interactions of the newer oral antifungal agents. Br J Dermatol 1999;141(Suppl 56): 26-32.
9. McInnes B D, Dockery G L. Surgical treatment of mycotic toenails. J Am Podiatr Med Assoc 1997;87:557-64.
10. Grover C, Bansal S, Nanda S, et al. Combination of surgical avulsion and topical therapy for single nail onychomycosis: a randomized controlled trial. Br J Dermatol 2007;157:364-8.
11. Ghannoum M A, Hajjeh R A, Scher R, et al. A large-scale North American study of fungal isolates from nails: the frequency of onychomycosis, fungal distribution, and antifungal susceptibility patterns. J Am Acad Dermatol 2000;43:641-8.
12. Huang Y Y, Chen A C, Carroll J D, Hamblin M R. Biphasic dose response in low level light therapy. Dose Response. 2009 September 1;7(4):358-83.

What is claimed is:

1. A method for treating fungal infection, such as onychomycosis, comprising irradiating a location with fungal infection with visible light, wherein said visible light could be 1) a minimum wavelength of about 400 nm and a maximum wavelength of about 500 nm, or 2) a minimum wavelength of about 600 nm and a maximum wavelength of about 700 nm, or 3) the combination of lights in both wavelength ranges described in 1) and 2) respectively.

2. The method of claim 1, wherein said visible light has a minimum average power density of 0.1 w/cm² and a maximum power density of 10 w/cm².

3. The method of claim 1, wherein the duration of irradiating said location with said visible light ranges from 1 minute to 120 minutes.

4. The method of claim 1, wherein said visible light has a minimum average power density of 0.1 w/cm² and a maximum average power density of 10 w/cm², and the duration of irradiating said location with said visible light ranges from 1 minute to 120 minutes.

5. The method of claim 1, wherein said visible light is produced by one or more light-emitting diode(s) or laser(s) or other light sources.

6. The system for treating fungal infection disease comprising means substantially as described in claim 1.

7. The system of claim 6, wherein said system further comprising:
a. a power supply module,
b. a control module, and
c. a treatment module.

8. The system of claim 7, wherein said power supply module comprises components that provide powers for said control module.

9. The system of claim 7, wherein said power supply module comprises components that provide powers for said treatment module.

10. The system of claim 7, wherein said power supply module comprises components that provide powers for said control module and said treatment module.

11. The system of claim 7, wherein said control module includes components that control said power supply module.

12. The system of claim 7, wherein said control module includes components that control said treatment module.

13. The system of claim 7, wherein said treatment module comprises one or more light-emitting diode(s) or laser(s) or other light source to provide specific treatment light in claim 1.

14. The system of claim 7, wherein said treatment module is designed in such a way as to hold said location with fungal infectious disease substantially in position.

15. The system of claim 7 further comprising modulating light delivery based on real-time temperature monitoring of the treatment site.

16. The method of claim 15, wherein temperature monitoring occurs through thermal imager or sensors.

17. The system of claim 15, wherein the controller is adapted to modulate the applied radiation in response to the temperature signal.

18. The system of claim 7, wherein said treatment module comprises a cooling system to maintain skin or other human body parts at a normal temperature.

19. The system of claim 18, wherein the cooling device includes a heat exchanger adapted to be positioned with a heat transfer surface adjacent to the treatment area which is in thermal communication with the heat exchanger.

20. The system of claim 19, wherein the control module controls the light generator and the cooling device whereby the control module responsive to the temperature signal to control the application of the light to the tissue by the light device and cooling of the treatment region whereby.
